(19) United States
(12) Patent Application Publication
(10) Pub. No.: US 2022/0142469 A1
(43) Pub. Date: May 12, 2022

(54) PATIENT POSITIONING APPARATUS FOR OPHTHALMIC DEVICES

(71) Applicant: Alcon Inc., Fribourg (CH)

(72) Inventors: Jeffrey W. Bentzler, Playa Del Rey, CA (US); Brian Bone, San Diego, CA (US); Andrew Zoolakis, Carlsbad, CA (US)

(21) Appl. No.: 17/501,844

(22) Filed: Oct. 14, 2021

Related U.S. Application Data
(60) Provisional application No. 63/112,946, filed on Nov. 12, 2020.

(51) Int. Cl. A61B 3/00

(52) U.S. Cl. CPC A61B 3/0083

(57) ABSTRACT

The present disclosure generally relates to patient positioning apparatuses for ophthalmic testing. In one or more embodiments, an ophthalmic device includes a base having a first side surface and an ophthalmic component disposed over or coupled to a top surface of the base. The ophthalmic component has a second side surface being separated by a distance in a first direction from the first side surface such that the ophthalmic component overhangs the base. The ophthalmic device includes a headrest assembly coupled to the base. The headrest assembly includes a first headrest assembly section extending from the first side surface of the base at least in part in the first direction, a second headrest assembly section extending from the first headrest assembly section at least in part in a second direction parallel to the second side surface, and a third headrest assembly section extending from the second headrest assembly section.

### BACKGROUND

### Field

Embodiments of the present disclosure generally relate to devices for ophthalmic testing, and more particularly, to patient positioning apparatuses therefor. Ophthalmic testing generally refers to the application of any ophthalmic diagnostic or imaging device for diagnosis, imaging, measurement, or other data acquisition pertaining to the eye.

### Description of the Related Art

Ophthalmic testing requires precise patient positioning and requires keeping the patient still to generate accurate results. In particular, precise ocular alignment and working distance must be set and maintained throughout the procedure. However, typical ophthalmic devices provide inadequate space for accommodating the torso of a patient. Furthermore, typical ophthalmic devices require the patient to lean in toward the device, which makes it difficult for the patient to remain still. These shortcomings can cause particular strain and discomfort for many patients including those having a condition making positioning difficult, such as those suffering from a fused neck or other spinal issues, back problems, tremors, or weakness.

FIG. 1 is a left side view of a prior art ophthalmic device 10. The ophthalmic device 10 generally includes a base 12 and an ophthalmic component 14 disposed on the base 12. A pair of headrest support arms 16 extend up from the base 12. The pair of headrest support arms 16 support a headrest 26 for positioning a head 22 of a patient 20 there against. The base 12 is disposed on a support surface 18, here a tabletop. As illustrated in FIG. 1, the patient 20 is forced to lean in toward the ophthalmic device 10 in order for the head 22 to be positioned against the headrest 26 (e.g., for the patient's chin to be positioned on a chin support 28, which is disposed below the headrest 26), which is required for establishing proper ocular alignment and working distance. In the process of leaning in, the torso 24 is forced into contact with the base 12, the headrest support arms 16, and the support surface 18. Thus, the patient is subjected to significant strain and discomfort which interferes with the ability of the patient to achieve and maintain proper positioning. Ultimately, incorrect patient positioning has a negative impact on the accuracy of the test results.

Therefore, what is needed in the art are improved patient positioning apparatuses for ophthalmic testing.

### SUMMARY

The present disclosure generally relates to devices for ophthalmic testing, and more particularly, to patient positioning apparatuses therefor.

In certain embodiments, an ophthalmic device is provided. The ophthalmic device includes a base having a first side surface and an ophthalmic component disposed over or coupled to a top surface of the base. The ophthalmic component has a second side surface being separated by a distance in a first direction from the first side surface such that the ophthalmic component overhangs the base. The ophthalmic device includes a headrest assembly coupled to the base. The headrest assembly includes a first headrest assembly section extending from the first side surface of the base at least in part in the first direction, a second headrest assembly section extending from the first headrest assembly section at least in part in a second direction parallel to the second side surface, and a third headrest assembly section extending from the second headrest assembly section, wherein the third headrest assembly section is shaped to receive a forehead of a patient.

In some other embodiments, a patient positioning apparatus for an ophthalmic component is provided. The patient positioning apparatus includes a base having a top surface configured to receive the ophthalmic component and a first side surface configured to be spaced from a second side surface of the ophthalmic component when the ophthalmic component is received by the top surface. The second side surface is separated by a distance in a first direction from the first side surface such that the ophthalmic component overhangs the base when the ophthalmic component is received by the top surface. The patient positioning apparatus includes a headrest assembly coupled to the base. The headrest assembly includes a first headrest assembly section extending from the first side surface of the base at least in part in the first direction, a second headrest assembly section extending from the first headrest assembly section at least in part in a second direction parallel to the first side surface and a third headrest assembly section extending from the second headrest assembly section, wherein the third headrest assembly section is shaped to receive a forehead of a patient.

In one or more embodiments, a headrest assembly for use with an ophthalmic component is provided. The headrest assembly includes a first headrest assembly section configured to be coupled to and extend from a base of the ophthalmic component at least in part in a first direction and at a first angle relative to the base, a second headrest assembly section extending from the first headrest assembly section at least in part in a second direction and at a second angle relative to the first headrest assembly section, and a third headrest assembly section extending from the second headrest assembly section, wherein the third headrest assembly section is shaped to receive a forehead of a patient.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a left side view of a prior art ophthalmic device, according to one or more embodiments.

FIG. 2A is a top isometric view of a front side of an exemplary ophthalmic device, according to one or more embodiments.

FIGS. 2B-2D are left side, front, and top views, respectively, of the ophthalmic device of FIG. 2A, according to one or more embodiments.

FIG. 2E is a left side view of the ophthalmic device of FIG. 2A illustrating a patient positioned therewith, according to one or more embodiments.

FIG. 3A is a left side elevation view of another exemplary ophthalmic device, according to one or more embodiments.

FIGS. 3B-3C are top isometric views of a front side of the ophthalmic device of FIG. 3A illustrating a chin support in raised and lowered positions, respectively.

FIG. 4 is a left side elevation view of another exemplary headrest assembly and chin support, according to one or more embodiments.

FIG. 5 is a left side elevation view of yet another exemplary headrest assembly and chin support, according to one or more embodiments.

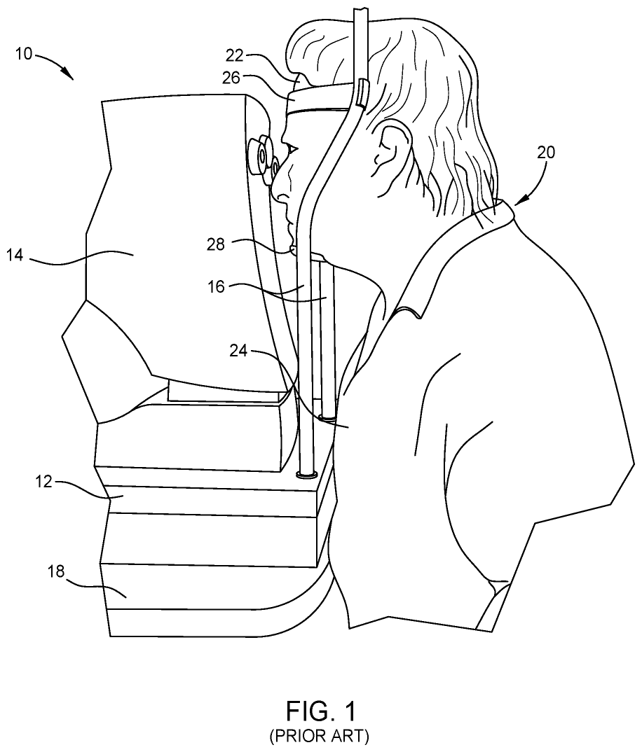

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally relates to devices for ophthalmic testing, and more particularly, to patient positioning apparatuses therefor.

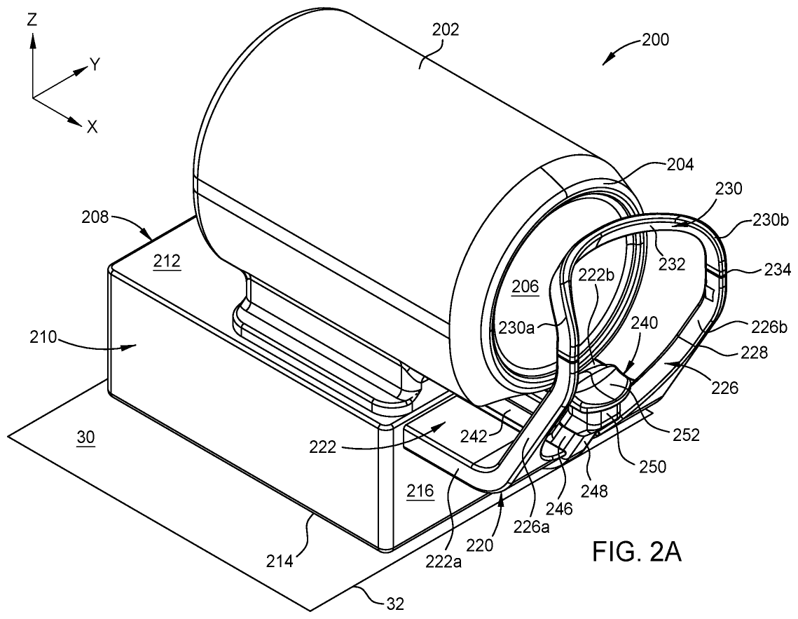

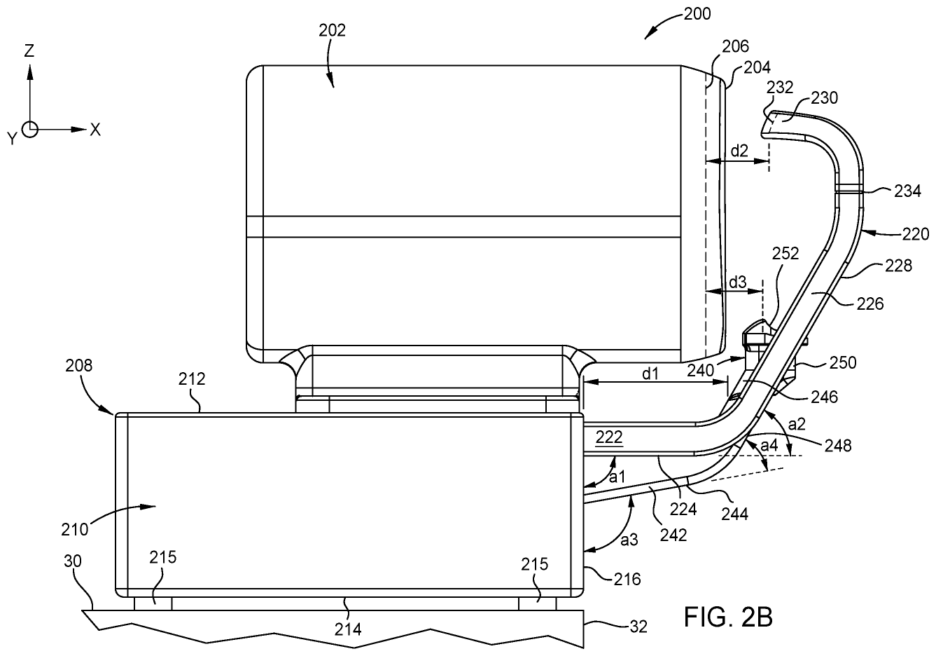

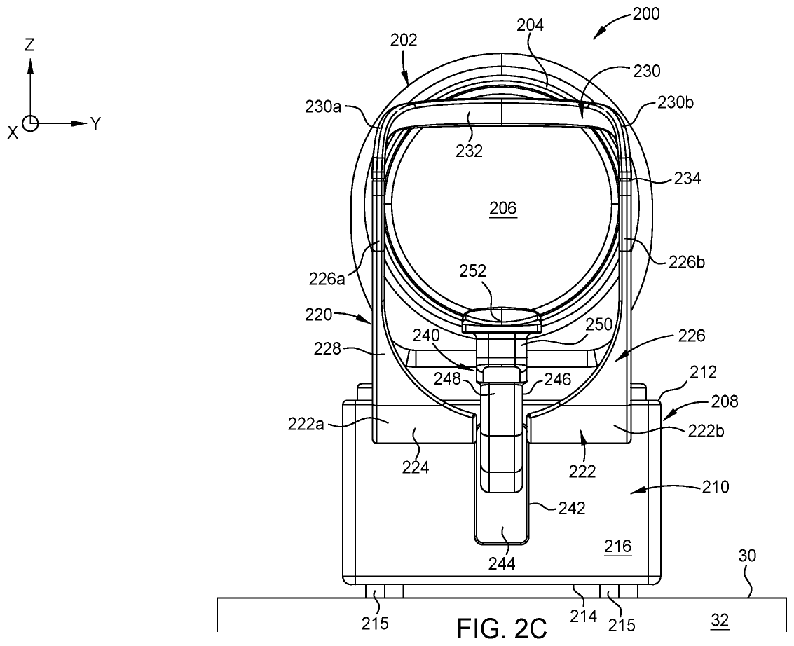

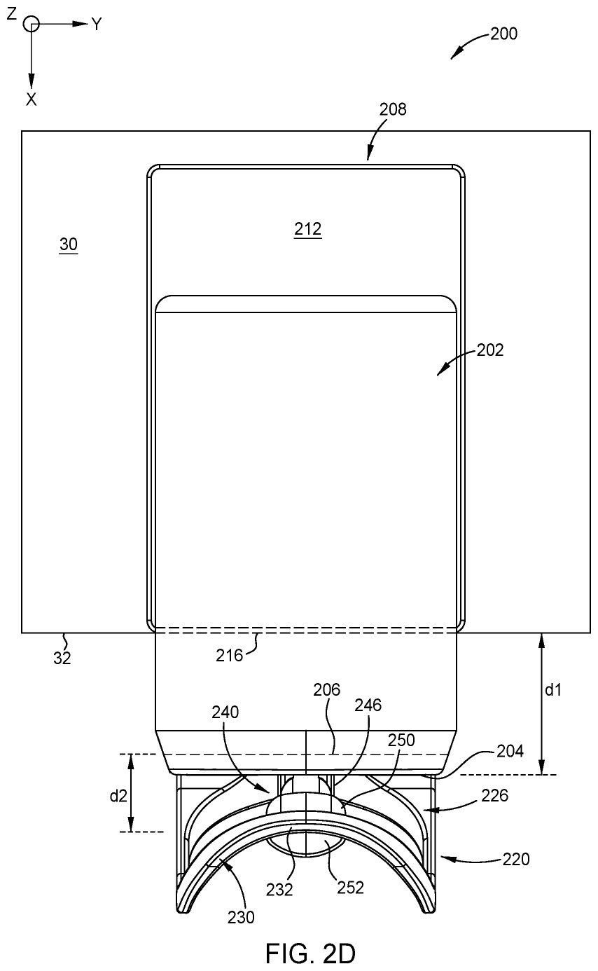

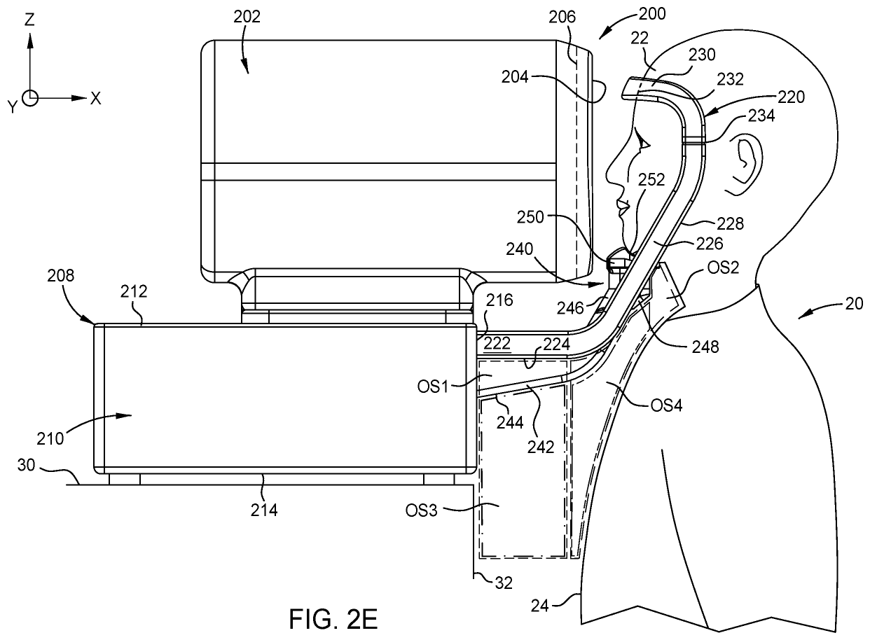

FIG. 2A is a top isometric view of a front side of an exemplary ophthalmic device 200, according to one or more embodiments. FIGS. 2B-2D are left side, front, and top views, respectively, of the ophthalmic device 200 of FIG. 2A. FIG. 2E is an alternative left side view of the ophthalmic device 200 of FIG. 2A illustrating a patient 20 positioned therewith. FIGS. 2A-2E are, therefore, described together herein for clarity.

The ophthalmic device 200 generally includes an ophthalmic component 202 and a patient positioning apparatus 208 configured to receive the ophthalmic component 202. The ophthalmic component 202 can be or include one or more ophthalmic testing devices, including without limitation diagnostic or imaging devices, e.g., an optical biometer, an optical coherence tomography (OCT) instrument such as a swept source-OCT (SS-OCT) biometer, an OCT ophthalmoscope, a camera for taking images of the eye, an instrument for taking measurements of the eye for diagnosis or pre-operative planning such as for intraocular lens (IOL) surgery, an instrument for generating data about the eye, a keratometer, an autorefractor, topography measurement devices, or combinations thereof.

The patient positioning apparatus 208 generally includes a base 210 for receiving and supporting the ophthalmic component 202, a headrest assembly 220 coupled to the base 210 for stabilizing a patient head 22 (FIG. 2E), and a chin support 240 coupled to the base 210 for supporting the patient head 22 (FIG. 2E). In the embodiments of FIGS. 2A-2E, the headrest assembly 220 and chin support 240 are separate components, each being independently coupled to the base 210. The headrest assembly 220 is fixedly coupled to the base 210 while the position of the chin support 240 may be adjusted relative to the base 210 and the headrest assembly 220.

Although in the embodiments of FIGS. 2A-2E, the headrest assembly 220 and chin support 240 are separate components, in certain embodiments (not shown), the headrest assembly 220 and chin support 240 are formed integrally with each other, with the base 210, or both. Also, in certain embodiments (not shown), the headrest assembly 220 and chin support 240 are coupled to the base 210 as a collective unit. Further, in certain embodiments, the positions of both the headrest assembly 220 and the chin support 240 may be adjusted relative to each other, relative to the base 210, or both. For example, the headrest assembly 220 and the chin support 240 may be independently adjustable where movement of the headrest assembly 220 is decoupled from movement of the chin support 240.

The base 210 has a top surface 212 for supporting the ophthalmic component 202 and a bottom surface 214 facing opposite the top surface 212. In some embodiments, the bottom surface 214 is configured to rest on a support surface 30. In the embodiments of FIGS. 2A-2E, a plurality of base legs 215 extend below the bottom surface 214 for directly contacting the support surface 30. In some other embodiments (not shown), the bottom surface 214 contacts the support surface 30 directly. The base 210 has a side surface 216 extending between the top and bottom surfaces 212, 214 and being located on a patient-facing side of the ophthalmic device 200. In the embodiments of FIGS. 2A-2E, the side surface 216 is oriented substantially in the direction of gravity (along the z-axis).

The ophthalmic component 202 is disposed over the top surface 212 of the base 210. In some embodiments, the ophthalmic component 202 is coupled to the base 210 by a secure attachment, e.g., one or more welds or fasteners, formed integrally with the base 210, held to the base 210 by gravity, or combinations thereof. The ophthalmic component 202 is configured to overhang an edge 32 of the support surface 30. In some embodiments, the support surface 30 is an elevated surface such as a tabletop. It will be appreciated that the base 210 can be sized in order to position the ophthalmic component 202 in ocular alignment with a patient depending on various factors including a height of the ophthalmic component 202, a height of the support surface 30, and a height of the patient 20 based on patient positioning, among other factors.

The ophthalmic component 202 has a side surface 204 located on the patient-facing side of the ophthalmic device 200. In the illustrated embodiments, the side surface 204 is oriented substantially in the direction of gravity (along the z-axis). In certain embodiments (not shown), the side surface 204 is inclined relative to the z-axis and may be at least partially aligned with one or more sections of the headrest assembly 220 or chin support 240. The side surface 204 is separated by a distance d1 in the +x direction from the side surface 216 of the base 210 (FIG. 2B). The ophthalmic component 202 overhangs each of the base 210 and the edge 32 in the +x direction. In some embodiments, the ophthalmic component 202 overhangs the edge 32 by about the distance d1 or less. In some embodiments, the distance d1 is about 4 inches or greater (e.g., from about 4 inches to about 12 inches, from about 4 inches to about 8 inches, from about 6 inches to about 8 inches, about 6 inches, or alternatively about 8 inches). In the embodiments of FIGS. 2A-2E, the side surface 216 of the base 210 and the side surface 204 of the ophthalmic component 202 are substantially parallel to each other, i.e., disposed in spaced apart substantially parallel planes. In some other embodiments, one or more faces and/or edges of each of the side surfaces 216, 204 are at least partially parallel to each other. In some other embodiments, the side surfaces 216, 204 are non-parallel.

The ophthalmic component 202 has a window 206 located on the patient-facing side of the ophthalmic device 200. In some embodiments, the window 206 is a protective covering. In some embodiments, the window 206 is or exposes a lens or some other testing, diagnostic, or imaging apparatus of the ophthalmic component 202. In the illustrated embodiments, the window 206 is recessed in the −x direction from the side surface 204. In some other embodiments, the window 206 is flush with the side surface 204.

The headrest assembly 220 generally includes a pair of side supports or headrest support arms (e.g., side supports 222a-b, 226a-b, 230a-b), which support a headrest (e.g., third headrest assembly section 230). The headrest assembly 220 includes one or more sections extending at respective angles relative to the base 210. The headrest assembly 220 is shaped to extend upward from the base 210 to an area proximate the side surface 204 of the ophthalmic component 202 to facilitate proper spatial orientation of the patient head 22 for ocular alignment and examination via the window 206. The headrest assembly 220 is configured to contact at least the forehead of a patient 20 to stabilize the patient head 22 thereby minimizing movement relative to the ophthalmic component 202.

The headrest assembly 220 includes a first headrest assembly section 222 extending from the side surface 216 of the base 210. The first headrest assembly section 222 is formed from or refers to side supports 222a-b. The first headrest assembly section 222 and the side surface 216 define a first open space OS1 directly below the first headrest assembly section 222 (FIG. 2E). The first headrest assembly section 222 extends at least in part in the +x direction from the side surface 216 at a first angle a1 (FIG. 2B). As defined herein, the first angle a1 is an angle of attachment between a bottom edge 224 of the first headrest assembly section 222 and the side surface 216. In the embodiments of FIGS. 2A-2E, the first headrest assembly section 222 is orthogonal to the side surface 216. In some embodiments, the angle a1 between the side surface 216 and the first headrest assembly section 222 is about 90 degrees or more (e.g., from about 90 degrees to about 135 degrees, from about 90 degrees to about 120 degrees, or from about 90 degrees to about 105 degrees). In some embodiments, the first headrest assembly section 222 is orthogonal to the direction of gravity. In some other embodiments, the first headrest assembly section 222 is inclined in the +z direction relative to the x-y plane (horizontal) by about 30 degrees or less (e.g., from about 0 degrees to about 30 degrees) or alternatively by about 15 degrees or less (e.g., from about 0 degrees to about 15 degrees).

The headrest assembly 220 includes a second headrest assembly section 226 extending from the first headrest assembly section 222. The second headrest assembly section 226 is formed from or refers to side supports 226a-b. The first and second headrest assembly sections 222, 226 define a second open space OS2 directly below the second headrest assembly section 226 (FIG. 2E). The second headrest assembly section 226 extends at least in part in a direction different from the first headrest assembly section 222 at a second angle a2 (FIG. 2B). As defined herein, the second angle a2 is an angle of inclination of a bottom edge 228 of the second headrest assembly section 226 relative to the bottom edge 224 of the first headrest assembly section 222. In some embodiments, the second angle a2 is inclined relative to the first angle a1 by about 30 degrees to about 90 degrees (e.g., from about 45 degrees to about 90 degrees or from about 45 degrees to about 60 degrees). In other words, an angle between the side surface 216 and the second headrest assembly section 226 is equal to the sum of the first and second angles a1, a2. In some embodiments, the second headrest assembly section 226 is inclined in the +z direction relative to the x-y plane (horizontal) by about 30 degrees or more (e.g., from about 30 degrees to about 90 degrees, from about 45 degrees to about 90 degrees, or from about 60 degrees to about 90 degrees). In some embodiments (not shown), the second headrest assembly section 226 is oriented substantially parallel to the side surface 204. In certain embodiments (not shown), the second angle a2 is 0 degrees such that the first and second headrest assembly sections 222, 226 extend from the base 210 with a single angle a1.

The headrest assembly 220 includes a third headrest assembly section 230, referred to as a headrest, extending from the second headrest assembly section 226. The third headrest assembly section 230 includes side supports 230a-b that couple to side supports 226a-b. The third headrest assembly section 230 is shaped to receive a forehead of the patient 20. The third headrest assembly section 230 has a contact surface 232 for contacting the forehead of the patient 20. In some embodiments, the contact surface 232 is curved to at least partially conform to the shape of the forehead. The third headrest assembly section 230 is shaped to extend toward the ophthalmic component 202 such that the contact surface 232 is closer to the window 206 than an interface 234 between the second and third headrest assembly sections 226, 230. However, the shape of the third headrest assembly section 230 is not limited to the illustrated embodiments. In certain embodiments (not shown), the third headrest assembly section 230 may not extend toward the ophthalmic component 202, e.g., when the second headrest assembly section 226 is substantially aligned in the direction of gravity (along the z-axis). In some embodiments, the contact surface 232 is spaced in the x-direction from the window 206 by a distance d2 of from about 1.5 inches to about 3 inches to set a proper focal distance. In some embodiments, the first, second and third headrest assembly sections 222, 226, 230 are integrally formed. In some other embodiments, one or more of the first, second, or third headrest assembly sections 222, 226, 230 are coupled to adjacent ones of the headrest assembly sections by a secure attachment, e.g., one or more fasteners.

In the illustrated embodiments, each of the first, second, and third headrest assembly sections 222, 226, 230 is formed from a pair of side supports spaced apart in the y-direction. The side supports 222a, 222b corresponding to the first headrest assembly section 222 are spaced apart by a distance configured to accommodate the chin support 240 therebetween. The side supports 226a-b, 230a-b corresponding to the second and third headrest assembly sections 226, 230, respectively, are spaced apart by a distance configured to receive the patient head 22 therebetween. In some embodiments, the side supports 222a, 222b corresponding to the first headrest assembly section 222 are wider in the y-direction than the side supports 226a-b, 230a-b of the second and third headrest assembly sections 226, 230 to enhance overall structural strength and rigidity of the headrest assembly 220.

The chin support 240 includes a first chin support section 242 extending from the side surface 216 of the base 210. The first chin support section 242 and the side surface 216 define a third open space OS3 directly below the first chin support section 242 (FIG. 2E). The first chin support section 242 extends at least in part in the +x direction from the side surface 216 at a third angle a3 (FIG. 2B). As defined herein, the third angle a3 is an angle of attachment between a bottom edge 244 of the first chin support section 242 and the side surface 216. In some embodiments, the angle a3 between the side surface 216 and the first chin support section 242 is about 90 degrees or more (e.g., from about 90 degrees to about 135 degrees, from about 90 degrees to about 120 degrees, from about 90 degrees to about 105 degrees, or alternatively from about 105 degrees to about 120 degrees). In some embodiments, the first chin support section 242 is orthogonal to the direction of gravity. In some other embodiments, the bottom edge 244 of the first chin support section 242 is inclined in the +z direction relative to the x-y plane (horizontal) by about 30 degrees or less (e.g., from about 0 degrees to about 30 degrees, from about 0 degrees to about 15 degrees, or alternatively from about 15 degrees to about 30 degrees). In the embodiments of FIGS. 2A-2E, the angle a3 of the first chin support section 242 is greater than the angle a1 of the first headrest assembly section 222. In some other embodiments, the angle a3 is about equal to the angle a1.

The chin support 240 includes an optional second chin support section 246 extending from the first chin support section 242. The first and second chin support sections 242, 246 define a fourth open space OS4 directly below the second chin support section 246 (FIG. 2E). The second chin support section 246 extends at least in part in a direction different from the first chin support section 242 at a fourth angle a4 (FIG. 2B). As defined herein, the fourth angle a4 is an angle of inclination of a bottom edge 248 of the second chin support section 246 relative to the bottom edge 244 of the first chin support section 242.

In some embodiments, the fourth angle a4 is inclined relative to the third angle a3 by about 0 degrees to about 90 degrees (e.g., from about 0 degrees to about 45 degrees, from about 0 degrees to about 30 degrees, or from about 15 degrees to about 30 degrees). In other words, an angle between the side surface 216 and the second chin support section 246 is equal to the sum of the third and fourth angles a3, a4. In some embodiments, the second chin support section 246 is inclined in the +z direction relative to the x-y plane (horizontal) by about 30 degrees or more (e.g., from about 30 degrees to about 90 degrees, from about 45 degrees to about 90 degrees, or from about 60 degrees to about 90 degrees). In some embodiments, the second chin support section 246 is oriented substantially parallel to the second headrest assembly section 226. In some embodiments, the second chin support section 246 is oriented substantially parallel to the side surface 204.

The chin support 240 includes a third chin support section 250 extending from the second chin support section 246. The third chin support section 250 is configured to receive a chin of a patient 20. The third chin support section 250 has a contact surface 252 for contacting the chin of the patient 20. In some embodiments, the contact surface 252 is curved to at least partially conform to the shape of the chin. The contact surface 252 is centered with the ophthalmic component 202 in the y-direction for centering the head 22 of the patient 20 relative thereto. In some embodiments, the contact surface 252 is spaced in the x-direction from the window 206 by a distance d3 of from about 1.5 inches to about 3 inches. In some embodiments, the distance d3 is about equal to the distance d2 so as to align the forehead and chin of the patient 20 along the y-z plane (vertical). In some embodiments, the first, second and third chin support sections 242, 246, 250 are integrally formed. In some other embodiments, one or more of the first, second, or third chin support sections 242, 246, 250 are coupled to adjacent ones of the chin support sections by a secure attachment, e.g., one or more fasteners.

In certain embodiments of FIGS. 2A-2E, the chin support 240 is movable, e.g., the chin support 240 can be raised and lowered. In certain embodiments, the base 210 includes an actuator (not shown), e.g., an electromechanical motor, for adjusting the position of the chin support 240. In certain embodiments, the base 210 includes a controller (not shown) with a processor and a memory. The memory stores instructions that when executed by the processor configure the controller to receive user input to adjust the position of the chin support 240. Based on the user input, the controller causes the actuator to physically adjust the position of the chin support 240. In certain embodiments, the actuator moves the chin support 240 substantially in the z-direction (vertical). Movement of the chin support 240 substantially along the z-axis maintains alignment in the y-z plane (vertical) between the forehead contact surface 232 and the chin contact surface 252. In certain embodiments, the chin support 240 moves parallel to the side surface 204. Beneficially, the chin support 240 being adjustable improves patient positioning by being adaptable to different head sizes. For pediatric patients and other patients with shorter faces, the chin support 240 can be moved to a raised position which shortens the distance between the forehead contact surface 232 and the chin contact surface 252. Alternatively, for patients with longer faces, the chin support 240 can be moved to a lowered position which lengthens the distance between the forehead contact surface 232 and the chin contact surface 252. In certain embodiments (not shown), the chin support 240 is also movable in the x-direction. Providing a custom setup for each patient by adjusting the chin support 240 ultimately improves patient comfort and accuracy of the test results. Moreover, in certain embodiments, the angles a3, a4 which define the profile of the chin support 240 are selected to enable the chin contact surface 252 to be raised and lowered without causing the chin support 240 to contact the torso 24. In other words, the open spaces OS3, OS4 between the chin support 240 and the torso 24 are maintained even when the chin support 240 is in the fully lowered position.

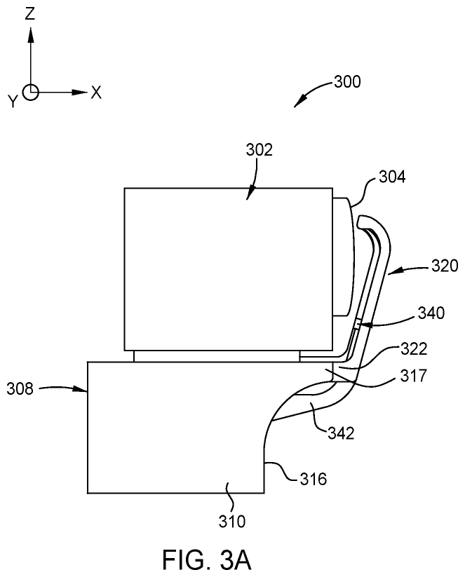

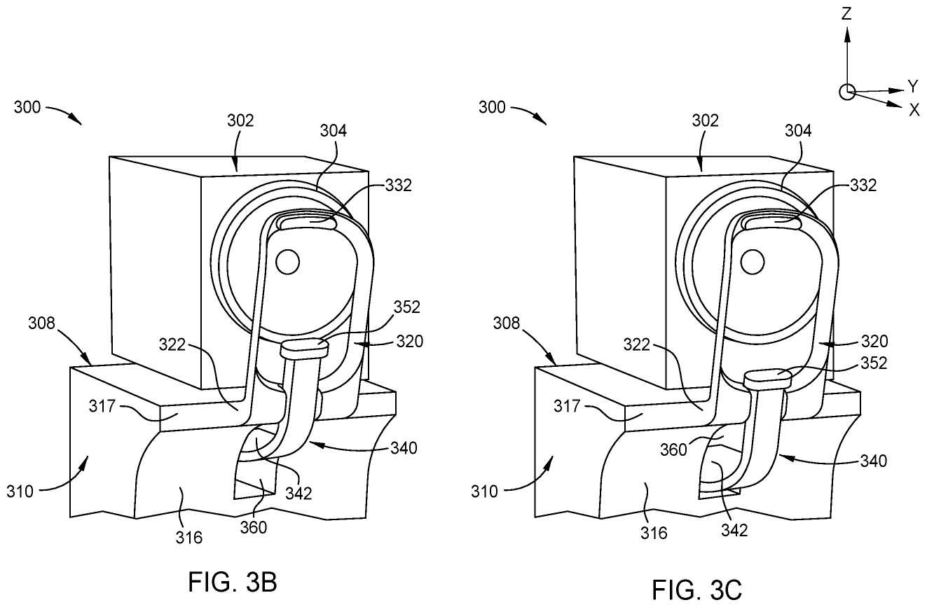

FIG. 3A is a left side elevation view of another exemplary ophthalmic device 300, according to one or more embodiments. The ophthalmic device 300 is similar to the ophthalmic device 200 of FIG. 2A except where noted, and corresponding description thereof may be incorporated herein without limitation. The ophthalmic device 300 generally includes an ophthalmic component 302 and a patient positioning apparatus 308 configured to receive the ophthalmic component 302. The ophthalmic component 302 has a side surface 304 located on the patient-facing side of the ophthalmic device 300. The patient positioning apparatus 308 generally includes a base 310 for receiving and supporting the ophthalmic component 302, a headrest assembly 320 coupled to the base 310 for stabilizing the patient head 22, and a chin support 340 coupled to the base 310 for supporting the patient head 22.

In the embodiments of FIG. 3A, the base 310 has a curved side surface 316. The curved side surface 316 is at least partially inclined relative the z-axis. In certain embodiments, the curved side surface 316 extends at least in part in a direction parallel to one or more sections of the headrest assembly 320 or chin support 340. An upper portion 317 of the curved side surface 316 extends in the +x direction by a distance, e.g., to about a plane corresponding to the side surface 304. In the embodiments of FIG. 3A, a first headrest assembly section 322 extends from the upper portion 317 of the curved side surface 316. In the embodiments of FIG. 3A, the first open space is formed directly below the curved side surface 316 and the first headrest assembly section 322. The first headrest assembly section 322 is shortened in the +x direction relative to the first headrest assembly section 222 of the ophthalmic device 200 of FIG. 2A.

In the embodiments of FIG. 3A, the chin support 340 extending from the curved side surface 316 is movable between a raised position (FIG. 3B) and a lowered position (FIG. 3C). In such embodiments, a notch 360 is formed in the curved side surface 316. A first chin support section 342 is disposed in the notch 360 and movably coupled to an actuator (not shown) for moving the chin support 340. The chin support 340 is constructed and arranged similar to the chin support 240 of FIGS. 2A-2E and corresponding description thereof may be incorporated herein without limitation.

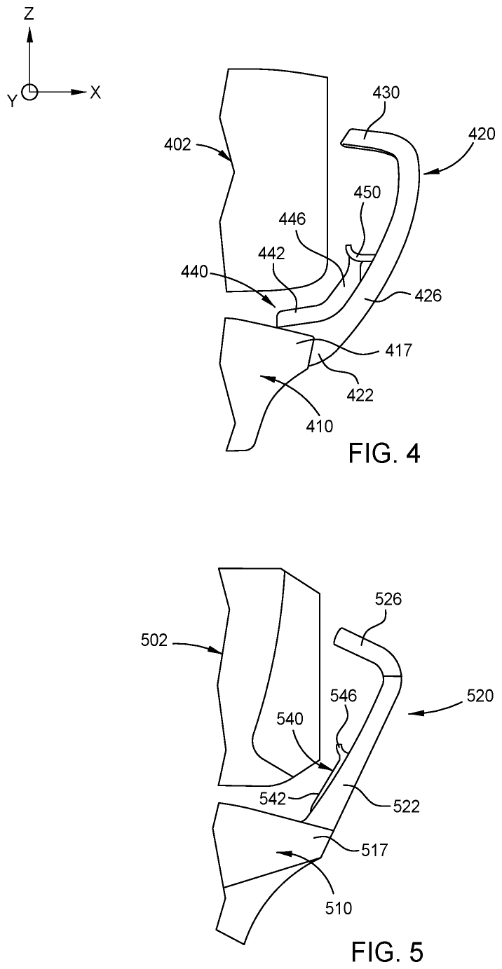

FIGS. 4-5 are left side elevation views of a curved headrest assembly 420 and chin support 440 and an angled headrest assembly 520 and chin support 540, respectively, according to one or more embodiments. The headrest assemblies 420, 520 are similar to the headrest assembly 220 of FIG. 2A except where noted. Likewise, the chin supports 440, 540 are similar to the chin support 240 of FIG. 2A except where noted. Referring to FIG. 4, the curved headrest assembly 420 and chin support 440 extend from an upper portion 417 of a base 410 which supports an ophthalmic component 402 thereabove. In certain embodiments of FIG. 4, first and second headrest assembly sections 422, 426 of the curved headrest assembly 420 each have a curved shape along a length thereof. In some embodiments, the curved headrest assembly 420 may more precisely conform to a patient. A third headrest assembly section 430 extends from the second headrest assembly section 426. First and second chin support sections 442, 446 of the curved chin support 440 each have a curved shape along a length thereof. A third chin support section 450 extends from the second chin support section 446. In some embodiments, the shape of the curved chin support 440 is conformal to the shape of the curved headrest assembly 420 to optimize the open spaces there below. Referring to FIG. 5, the angled headrest assembly 520 and chin support 540 extend from an upper portion 517 of a base 510 which supports an ophthalmic component 502 thereabove. In the embodiments of FIG. 5, the angled headrest assembly 520 includes only two headrest assembly sections which are constructed and arranged similar to the second and third headrest assembly sections 226, 230 of FIG. 2A. A first headrest assembly section 522 extending from the upper portion 517 of the base 510 is substantially straight. A second headrest assembly section 526 extends from the first headrest assembly section 522. Likewise, the angled chin support 540 includes only two chin support sections which are constructed and arranged similar to the second and third chin support sections 242, 246 of FIG. 2A. A first chin support section 542 extending from the upper portion 517 is substantially straight. A second chin support section 546 extends from the first chin support section 542. In some embodiments, the shape of the angled chin support 540 is conformal to the shape of the angled headrest assembly 520 to optimize the open spaces there below.

In summary, embodiments of the present disclosure improve patient positioning for ophthalmic testing. In particular, embodiments of the present disclosure provide adequate space (e.g., the open spaces shown in the Figures) for accommodating the torso of a patient and reduce patient lean in toward the testing device. Because of these changes, the patient is relieved of significant strain and discomfort which interferes with the ability of the patient to achieve and maintain proper positioning, including precise ocular alignment and working distance, throughout the procedure. Ultimately, correcting patient positioning improves accuracy of the test results.

While the foregoing is directed to embodiments of the present disclosure, other and further embodiments of the disclosure may be devised without departing from the basic scope thereof, and the scope thereof is determined by the claims that follow.

### Example Embodiments

Embodiment 1: The ophthalmic device of claim 1, wherein the first headrest assembly section extends from the base with a first angle, and wherein the second headrest assembly section extends from the first headrest assembly section with a second angle different from the first angle.

Embodiment 2: The ophthalmic device of embodiment 1, wherein the first angle is inclined relative to horizontal by 15 degrees or less, and wherein the second angle is inclined relative to the first angle by 30 degrees to 90 degrees.

Embodiment 3: The ophthalmic device of claim 1, wherein at least one of the first, second, and third headrest assembly sections comprises spaced apart side supports.

Embodiment 4: The ophthalmic device of embodiment 3, wherein the third headrest assembly section is spaced in the first direction from the second side surface by 1.5 inches to 3 inches.

Embodiment 5: The patient positioning apparatus of claim 10, the chin support further comprising: a third chin support section extending from the first chin support section at least in part in the second direction parallel to the first side surface.

Embodiment 6: The patient positioning apparatus of embodiment 5, wherein the third chin support section and the first chin support section define an open space directly below the third chin support section.

Embodiment 7: The patient positioning apparatus of claim 9, wherein the first headrest assembly section extends from the base with a first angle, and wherein the second headrest assembly section extends from the first headrest assembly section with a second angle different from the first angle.

Embodiment 8: The patient positioning apparatus of embodiment 7, wherein the first angle is inclined relative to horizontal by 15 degrees or less, and wherein the second angle is inclined relative to the first angle by 30 degrees to 90 degrees.

Embodiment 9: The patient positioning apparatus of claim 9, wherein at least one of the first, second, and third headrest assembly sections comprises spaced apart side supports.

Embodiment 10: The patient positioning apparatus of claim 9, wherein the base is integral with the ophthalmic component.

What is claimed is:

1. An ophthalmic device, comprising:
a base having a first side surface;
an ophthalmic component disposed over or coupled to a top surface of the base, the ophthalmic component having a second side surface being separated by a distance in a first direction from the first side surface such that the ophthalmic component overhangs the base; and
a headrest assembly coupled to the base, the headrest assembly comprising:
a first headrest assembly section extending from the first side surface of the base at least in part in the first direction;
a second headrest assembly section extending from the first headrest assembly section at least in part in a second direction parallel to the second side surface; and
a third headrest assembly section extending from the second headrest assembly section, wherein the third headrest assembly section is shaped to receive a forehead of a patient.

2. The ophthalmic device of claim 1, further comprising a chin support coupled to the base, the chin support comprising:
a first chin support section extending from the first side surface of the base; and
a second chin support section configured to receive a chin of the patient.

3. The ophthalmic device of claim 2, wherein the first chin support section and the first side surface of the base define an open space directly below the first chin support section.

4. The ophthalmic device of claim 2, the chin support further comprising:
a third chin support section extending from the first chin support section at least in part in the second direction parallel to the second side surface.

5. The ophthalmic device of claim 4, wherein the third chin support section and the first chin support section define an open space directly below the third chin support section.

6. The ophthalmic device of claim 1, wherein:
the first headrest assembly section and the first side surface define a first open space directly below the first headrest assembly section; and
the second headrest assembly section and the first headrest assembly section define a second open space directly below the second headrest assembly section.

7. The ophthalmic device of claim 1, wherein the distance between the first and second side surfaces in the first direction is in a range of 4 inches to 12 inches.

8. The ophthalmic device of claim 1, wherein the ophthalmic device is configured to rest on a support surface, and wherein the ophthalmic component is configured to overhang an edge of the support surface in the first direction.

9. A patient positioning apparatus for an ophthalmic component, comprising:
a base comprising:
a top surface configured to receive the ophthalmic component; and
a first side surface configured to be spaced from a second side surface of the ophthalmic component when the ophthalmic component is received by the top surface, the second side surface being separated by a distance in a first direction from the first side surface such that the ophthalmic component overhangs the base when the ophthalmic component is received by the top surface; and
a headrest assembly coupled to the base, the headrest assembly comprising:
a first headrest assembly section extending from the first side surface of the base at least in part in the first direction;
a second headrest assembly section extending from the first headrest assembly section at least in part in a second direction parallel to the first side surface; and
a third headrest assembly section extending from the second headrest assembly section, wherein the third headrest assembly section is shaped to receive a forehead of a patient.

10. The patient positioning apparatus of claim 9, further comprising a chin support, the chin support comprising:
a first chin support section extending from the first side surface of the base; and
a second chin support section configured to receive a chin of the patient.

11. The patient positioning apparatus of claim 10, wherein the first chin support section and the first side surface of the base define an open space directly below the first chin support section.

12. The patient positioning apparatus of claim 9, wherein:
the first headrest assembly section and the first side surface define a first open space directly below the first headrest assembly section; and
the second headrest assembly section and the first headrest assembly section define a second open space directly below the second headrest assembly section.

13. A headrest assembly for use with an ophthalmic component the headrest assembly comprising:
a first headrest assembly section configured to be coupled to and extend from a base of the ophthalmic component at least in part in a first direction and at a first angle relative to the base;
a second headrest assembly section extending from the first headrest assembly section at least in part in a second direction and at a second angle relative to the first headrest assembly section; and
a third headrest assembly section extending from the second headrest assembly section, wherein the third headrest assembly section is shaped to receive a forehead of a patient.

14. The headrest assembly of claim 13, wherein the first angle is substantially orthogonal to the direction of gravity.

15. The headrest assembly of claim 13, wherein the second angle is inclined relative to the first angle by 45 degrees to 90 degrees.
